(19) United States
(12) Patent Application Publication
(10) Pub. No.: US 2026/0060667 A1
(43) Pub. Date: Mar. 5, 2026

(54) Soft Anchor Constructs and Methods of Tissue Repairs

(71) Applicant: Arthrex, Inc., Naples, FL (US)

(72) Inventors: Peter J. DREYFUSS, Naples, FL (US); Gautam P. YAGNIK, Miami, FL (US); Christopher W. HODGKINS, Coral Gables, FL (US)

(73) Assignee: Arthrex, Inc., Naples, FL (US)

(21) Appl. No.: 19/283,299

(22) Filed: Jul. 29, 2025

Related U.S. Application Data
(60) Provisional application No. 63/687,819, filed on Aug. 28, 2024.

(51) Int. Cl. A61B 17/04; A61B 17/00; A61B 17/56; A61B 17/68

(52) U.S. Cl. CPC A61B 17/0401; A61B 17/68; A61B 2017/00862; A61B 2017/0404; A61B 2017/0445; A61B 2017/0459; A61B 2017/564

(57) ABSTRACT

Surgical anchor constructs, fixation devices, and methods for tissue repairs are disclosed. A soft suture anchor has a cannulated sheath (that is fixed in bone) and at least one flexible strand attached to the sheath. Free ends of the flexible strand are passed through the lumen and the body of the sheath in different directions and for multiple times, to each form at least one splice and a plurality of adjustable, flexible, tensionable, knotless loops extending around and through the cannulated sheath.

### CROSS-REFERENCE TO RELATED APPLICATIONS

This application claim priority to U.S. Provisional Application No. 63/687,819 filed on Aug. 28, 2024, the disclosure of which is hereby incorporated by reference in its entirety herein.

### BACKGROUND

The disclosure relates to the field of surgery and, more specifically, to surgical suture constructs and methods of making, and tissue repairs for reconstructive surgeries.

### SUMMARY

Surgical flexible constructs, fixation devices, methods of making, and methods of tissue repairs are disclosed.

A surgical construct is a soft anchor with a sheath (that is fixed in bone) and at least one flexible strand attached to the sheath. A flexible strand can be suture that is passed through the lumen of the sheath in different directions and for a predetermined number of times to form at least two splices and a plurality of knotless loops. A soft anchor sheath can be a suture sheath.

A surgical construct allows improved fixation strength of soft anchors in less quality bone by causing the soft anchor to be shaped more like a washer when tension is applied to the construct during the technique. A surgical construct can create a tensionable, self-locking, knotless, reinforced repair. A surgical construct can be employed in knotless fixation of first tissue to second tissue, for example, fixation of soft tissue to bone.

Methods of tissue repairs are also disclosed. A surgical construct can provide tissue to tissue fixation with fewer passing steps, and with increased fixation and soft tissue compression. A tissue fixation can be a knotless repair. The unique passing of the suture limbs of the construct through a soft anchor allows the anchor to bunch up in a specific shape (more oval and/or circular) to allow increased strength of knotless repairs, such as syndesmosis-type repairs.

### DESCRIPTION OF THE DRAWINGS

FIG. 1 illustrates a surgical construct.

FIG. 2 illustrates another surgical construct.

FIG. 3 illustrates the surgical construct of FIG. 1 in a non-tensioned state.

FIG. 4 illustrates the surgical construct of FIG. 1 in a tensioned state.

FIGS. 5 and 6 illustrate an exemplary repair with surgical constructs.

FIGS. 7 and 8 illustrate another exemplary repair with surgical constructs.

### DETAILED DESCRIPTION

The disclosure provides surgical fixation devices, constructs, methods for manufacturing, and tissue repairs and reconstructions.

A soft anchor construct includes a sheath and at least one flexible strand attached to the sheath. The at least one flexible strand can be suture that has free ends passed through a lumen of the sheath in a specific direction and configuration to form a plurality of adjustable, flexible, tensionable loops through and around the sheath. A soft anchor construct can be a knotless construct. A soft anchor construct can be employed in AC repairs or syndesmosis-type repairs, among many others.

A soft anchor construct includes a cannulated sheath with a flexible coupler (suture) that passes through a lumen of the sheath and enters/exits the lumen of the sheath for different times and in different directions to form a plurality of splices and adjustable, flexible, tensionable loops. The adjustable, flexible, tensionable loops can be knotless.

The soft anchor sheath can consist essentially of suture. A surgical construct can create a self-tensioning, self-locking, tensionable, knotless, reinforced repair. A surgical construct can be employed in fixation of first tissue to second tissue, for example, syndesmosis repairs.

Methods of tissue repairs with soft anchor constructs are also disclosed. An exemplary method includes inter alia the steps of: (i) passing free ends of a flexible strand through a lumen and a body of a cannulated soft anchor sheath so that each free end of the flexible strand enters/exits the lumen and the body of the sheath a plurality of times to form a splice and at least one adjustable, flexible, tensionable loop around and through the sheath; and (ii) employing the sheath for one or more tissue repair applications. The one or more tissue repair applications can include soft tissue repairs; orthopedic surgical repairs such as rotator cuff repairs, Achilles tendon repairs, patellar tendon repairs, ACL/PCL reconstructions, AC repairs, hip and shoulder reconstructions, and hand, wrist, foot, and ankle repairs, among many others. The one or more tissue repairs can include a knotless repair.

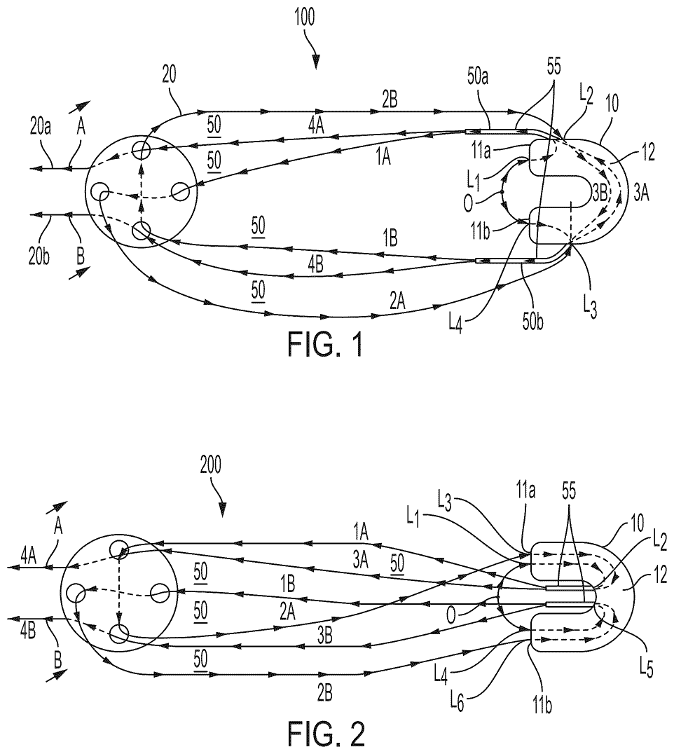

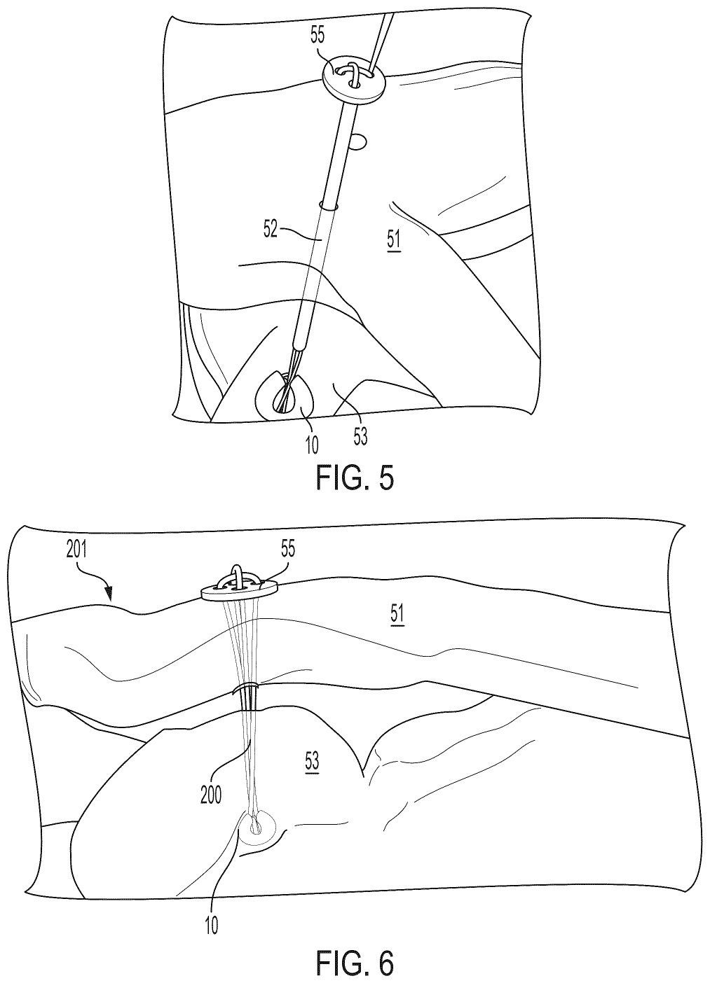

Referring now to the drawings, where like elements are designated by like reference numerals, FIGS. 1-4 illustrate exemplary fixation devices 100, 200 of the present disclosure. Fixation devices 100, 200 can be soft anchors, soft anchor constructs, soft suture anchors with a novel suture configuration, all-suture anchors, surgical constructs, and/or knotless anchor constructs. FIGS. 5-8 illustrate exemplary repairs 101, 201 with any of fixation devices 100, 200.

The fixation devices detailed below are soft anchors formed of “soft” materials, such as suture materials, that confer the ability to be inserted into bone sockets/holes/tunnels and bunch together, collapse, expand and/or change shape to fixate within the socket/hole/tunnel. In some embodiments, the soft anchor includes a cannulated sheath and one flexible strand attached to the sheath and forming a plurality of adjustable, flexible, tensionable loops. The flexible strand can be slidable relative to the sheath. In other embodiments, the soft anchor includes a sheath and a plurality of flexible strands, wherein some or all of the flexible strands are slidable relative to the sheath and forming at least one adjustable, flexible, tensionable loop. The soft anchors can be utilized in various surgical techniques to attach tissue to bone. The adjustable, flexible, tensionable loops can form an overall knotless construct.

FIG. 1 illustrates exemplary soft anchor 100 including cannulated sheath 10 and flexible strand 20. For a better understanding of the disclosure, left-side view of FIG. 1 illustrates a schematic view of the way the two ends of a flexible coupler pass through the sheath 10 of the soft anchor construct 100.

Sheath 10 can be in the form of a tubular sleeve or tubular member with a body made of a flexible material, such as a braided, woven, or knitted structure made of yarns, fibers, filaments, sutures or similar materials, or combinations of these materials. In one embodiment, sheath 10 is constructed of ultra-high molecular weight polyethylene (UHMWPE). In one embodiment, sheath 10 is constructed of UHMWPE and polyester. In another embodiment, sheath 10 is constructed of UHMWPE with an elastic component. In another embodiment, sheath 10 is constructed of polyester suture material with an elastic component. The elastic component can be elastane. The elastic component can be incorporated into the sheath via braiding, weaving, and/or knitting. In an embodiment, an elastic sheath can include a combination of elastic, polyester, and UHMWPE all braided in a tubular jacket. In an embodiment, an elastic sheath can consist essentially of elastane, polyester, and UHMWPE. The elastic component provides elasticity while the other components provide strength and limit the elongation of the suture.

Sheath 10 can also be any tubular or non-tubular structure having a lumen 12 along its length. The lumen can have a constant diameter. The lumen can have a varying diameter along its length.

Soft anchor 100 can include at least one flexible strand 20 passing through cannulated sheath 10. Sheath 10 includes a tubular body that extends between opposing ends 11a, 11b. The opposing ends 11a, 11b can be open or closed ends. The tubular body establishes bore or lumen 12 that extends between the opposing ends 11a, 11b. As shown in FIG. 1, free ends 20a, 20b of flexible strands 20 (flexible coupler 20; suture 20) are passed through portions of bore of the sheath 10 and through the body of the sheath 10, in a specific manner and direction and for a number of times, and as described in more detail below.

In an exemplary-only embodiment and as depicted in FIG. 1, “O” represents about the mid-length of flexible strand 20 with two free ends 20a, 20b. The trajectory of each of the two free ends 20a, 20b of the flexible strand 20 through the sheath 10 is illustrated by arrow A (for free end 20a) and arrow B (for free end 20b). Subsequent steps labeled 1A-4A are shown along arrow A for end 20a (4A); subsequent steps labeled 1B-4B are shown along arrow B for end 20b (4B).

End 20a of the flexible strand 20 follows the direction of arrow A, i.e., it is passed through the sheath 10 in the direction of arrow A. As such, free end 20a of the flexible strand 20 enters one of the ends 11a, 11b of the sheath 20 at a location L1, for example, end 11a; passes through at least a portion of lumen 12 of sheath 10 and exits at a location L2 (situated between the opposing ends 11a, 11b); reenters the sheath 20 at a location L3 (situated between the opposing ends 11a, 11b) forming a first adjustable, flexible, knotless, tensionable loop 50; passes through the lumen 12 of sheath 10; exits the sheath 20 at location L2 (or about location L2); and forms splice 50a and another adjustable, flexible, knotless, tensionable loop 50.

End 20b of the flexible strand 20 follows the direction of arrow B, i.e., it is passed through the sheath 10 in the direction of arrow B. Direction of arrow A is opposite direction of arrow B. As such, free end 20b of the flexible strand 20 enters the other one of the ends 11a, 11b of the sheath 20 at a location L4, for example, end 11b; passes through at least a portion of lumen 12 of sheath 10 and exits at location L3 (situated between the opposing ends 11a, 11b); reenters the sheath 20 at a location L2 (situated between the opposing ends 11a, 11b) to form an adjustable, flexible, knotless, tensionable loop 50; passes through the lumen 12 of sheath 10; exits the sheath 20 at location L3; and forms splice 50b and another adjustable, flexible, knotless, tensionable loop 50.

Each of splices 50a, 50b can be of about 10 mm. Ends 20a, 20b can be employed to tension the repair and can be cut to allow for a knotless repair.

Reference is now made to FIG. 2, which illustrates soft anchor 200 which is about similar to soft anchor 100 of FIG. 1 in that flexible strand 20 also forms two splices 50a, 50b and multiple adjustable, flexible, knotless, tensionable loops 50; however, soft anchor 200 differs from soft anchor 100 in the way ends 20a, 20b enter and exit the sheath 10 and as described below. “O” represents about the mid-length of flexible strand 20.

The trajectory of each of the two free ends 20a, 20b of the flexible strand 20 through the sheath 10 is illustrated by arrow A (for free end 20a) and arrow B (for free end 20b). Subsequent steps labeled 1A-4A are shown along arrow A for end 20a (4A); subsequent steps labeled 1B-4B are shown along arrow B for end 20b (4B).

End 20a of the flexible strand 20 follows the direction of arrow A, i.e., it is passed through the sheath 10 in the direction of arrow A. As such, free end 20a enters one of the ends 11a, 11b of the sheath 20 at a location L1, for example, end 11a; passes through at least a portion of lumen 12 of sheath 10 and exits at a location L2 (situated between the opposing ends 11a, 11b); reenters the sheath 20 at a location L3 at same end 11a, forming an adjustable, flexible, knotless, tensionable loop 50; passes through the lumen 12 of sheath 10; exits the sheath 20 at location L2; and forms splice 50a and another adjustable, flexible, knotless, tensionable loop 50 outside of the sheath. Locations L1 and L3 can coincide.

End 20b of the flexible strand 20 follows the direction of arrow B, i.e., it is passed through the sheath 10 in the direction of arrow B. Direction of arrow A is opposite direction of arrow B. As such, free end 20b enters the other one of the ends 11a, 11b of the sheath 20 (for example, end 11b) at a location L4; passes through at least a portion of lumen 12 of sheath 10 and exits at location L5 (situated between the opposing ends 11a, 11b); reenters the sheath 20 at a location L6 at the same end 11b to form an adjustable, flexible, knotless, tensionable loop 50; passes through the lumen 12 of sheath 10; exits the sheath 20 at location L5; and forms splice 50b and another adjustable, flexible, knotless, tensionable loop 50 outside of the sheath. Locations L4 and L6 can coincide.

Each of splices 50a, 50b can be of about 10 mm. Free ends 20a, 20b can be employed to tension the repair and can be cut to allow for a knotless repair.

Locations L1, L2, L3, L4, L45, L6 can be points of entry and/or exit in the form of openings formed through the sheath 10 (except for when they represent entry/exit through the open ends 11a, 11b). Locations L1, L2, L3, L4, L45, L6 can be pre-formed openings formed within and through the sheath 10 during the manufacturing process. Locations L1, L2, L3, L4, L45, L6 can be formed in situ, i.e., during the tissue repair by simply passing free ends 20a, 20b of the flexible strand 20 through the lumen 12 of the sheath and entering and exiting the sheath at the specific locations.

In an embodiment, and as shown in FIG. 1, locations L2, L3 are located about symmetric relative to transversal axis 13b of the sheath 10 and on a same side relative to longitudinal axis 13a of the sheath 10. In an embodiment, locations L2, L3 can be located asymmetric relative to transversal axis 13b of the sheath 10 and on a same side relative to longitudinal axis 13a of the sheath 10. In an embodiment, locations L2, L3 can be located about symmetric relative to transversal axis 13b of the sheath 10 and on different sides relative to longitudinal axis 13a of the sheath 10. In an embodiment, locations L2, L3 can be located asymmetric relative to transversal axis 13b of the sheath 10 and on different sides relative to longitudinal axis 13a of the sheath 10.

In an embodiment, and as shown in FIG. 2, locations L2, L5 are located about symmetric relative to transversal axis 13b of the sheath 10 and on a same side relative to longitudinal axis 13a of the sheath 10. In an embodiment, locations L2, L5 can be located asymmetric relative to transversal axis 13b of the sheath 10 and on a same side relative to longitudinal axis 13a of the sheath 10. In an embodiment, locations L2, L5 can be located about symmetric relative to transversal axis 13b of the sheath 10 and on different sides relative to longitudinal axis 13a of the sheath 10. In an embodiment, locations L2, L5 can be located asymmetric relative to transversal axis 13b of the sheath 10 and on different sides relative to longitudinal axis 13a of the sheath 10.

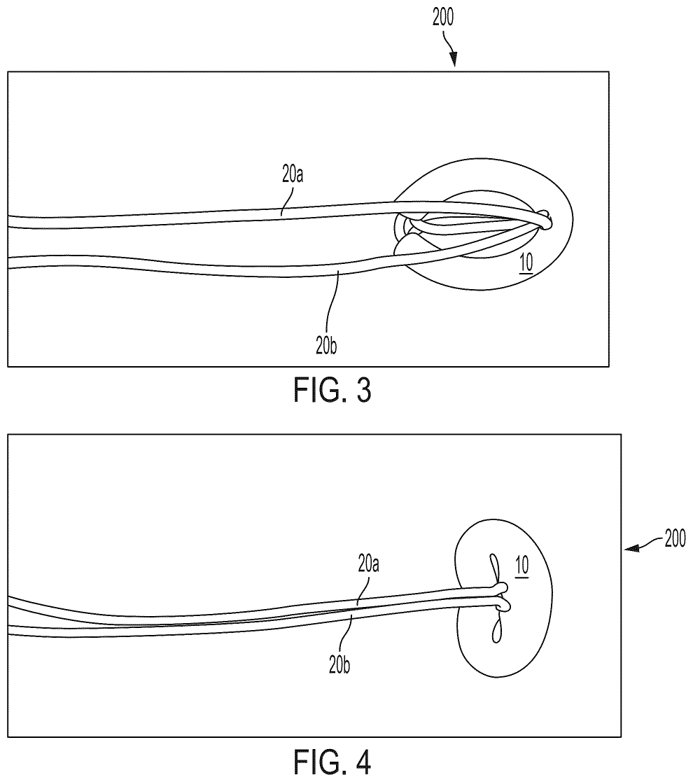

FIGS. 3 and 4 illustrate top views of surgical soft anchor construct 200 of FIG. 2. FIG. 3 shows soft anchor construct 200 in a non-tensioned (first configuration or relaxed configuration). FIG. 4 shows soft anchor construct 200 in a tensioned (second configuration or bunched configuration). Ends 20a, 20b of flexible strand 20 can assist in bunching together the sheath 10 once the soft anchor 200 is inserted into bone or secured over bone and once ends 20a, 20b the flexible strand 20 are tensioned. Ends 20a, 20b can be employed in additional fixation steps and/or can be cut (for a knotless repair) or can be knotted. The overall shape of the sheath 10 and soft anchor construct 200 becomes about oval, so the anchor acts more like a washer when tension is applied to the construct during the surgical technique, and as detailed below. The middle of the soft anchor construct 100, 200 becomes an integral part of the whole and, because of the unique passing of suture limbs 20a, 20b, the suture can continue to move once tension is applied.

A suture passing device (shuttling device; suture passer) such as a Nitinol loop can be attached to flexible free ends 20a, 20b of flexible strand 20 to aid in suture passing through the sheath and the splice formation.

Flexible strand 20 can be a suture. Non-limiting examples of suitable sutures include FiberWire®, TigerWire®, or FiberChain® suture, although any type of suture may be utilized, including cored or coreless sutures. In another embodiment, flexible strand 20 can include any soft, flexible strand of material.

Soft anchor 100, 200 is configured for use in various soft tissue repairs or fixations and can be fixated inside bone or over bone for attaching tissue (e.g., ligament, tendon, graft, etc.) to bone. For example, the soft anchor 100, 200 can be used in conjunction with a variety of orthopedic surgical repairs, including but not limited to rotator cuff repairs, AC repairs, Achilles tendon repairs, patellar tendon repairs, ACL/PCL reconstructions, hip and shoulder reconstructions, among many others. The fixation can be on or over bone. Particular applications of soft anchor 100, 200 are repairs of hand, wrist, foot, ankle and small joints for distal extremities, given the nano dimensions of the soft anchors.

Soft anchor 100, 200 is a “soft” construct because it is formed of soft materials such as yarns, fibers, filaments, strings, fibrils, strands, sutures, etc., or any combination of such materials. The soft materials may be synthetic or natural materials, or combinations of synthetic and natural materials, and may be bio-degradable or non-degradable, and may be elastic or non-elastic within the scope of this disclosure. In one non-limiting embodiment, the soft anchor 100, 200 is made exclusively of soft, suture-based materials. Soft anchor 100, 200 can be an “all-suture” construct.

FIGS. 5 and 6 illustrate an exemplary repair 201 (knotless acromioclavicular (AC) repair) with any of soft anchors 100, 200 detailed above. In an exemplary embodiment, soft anchors 100, 200 can provide reduction and stabilization of acute and chronic acromioclavicular joint separations. Any of soft anchors 100, 200 can be assembled or preassembled with a fixation device such as an implantable button, for example, a titanium round or oblong button or a Dog Bone™ button, which attaches to the suture loops of soft anchors 100, 200.

A tunnel 52 is drilled through clavicle 51 and coracoid 53 using a cannulated drill. A button 55 is attached to exemplary soft anchor 200. Sheath 10 is delivered to the coracoid base and button 55 is secured on the clavicle, as part of repair 201 (FIG. 6). In an embodiment, sheath 10 can be positioned on the coracoid base or within the coracoid. In an embodiment, sheath 10 can be attached to (or provided within) the clavicle and a button can be secured on the coracoid base. Repair 201 is a knotless repair.

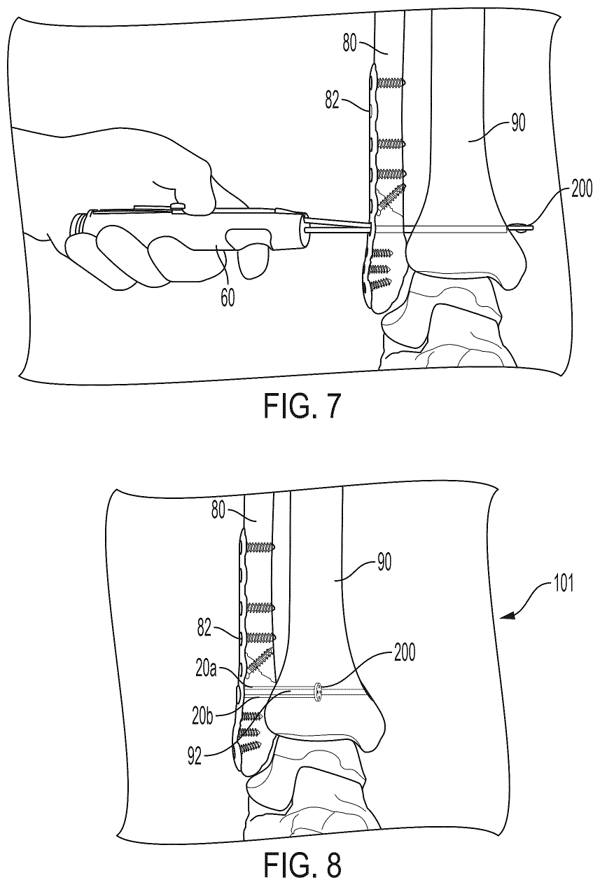

FIGS. 7 and 8 illustrate an exemplary repair 101 (syndesmosis repair) with any of soft anchors 100, 200 detailed above (which act as syndesmosis implants 100, 200). The syndesmosis soft anchor devices 100, 200 provide fixation during the healing process following a syndesmotic trauma, such as fixation of syndesmosis (syndesmosis disruptions) in connection with Weber B and C ankle fractures.

FIG. 7 illustrates exemplary fracture stabilization on fibula 80 with plate 82. Soft anchor construct 200 is secured to inserter 60 and advanced through the fibula 80 and through tibia bone tunnel 92 formed within tibia 90.

Soft anchor construct 200 is positioned at about mid-length of tibial tunnel 92, as shown in FIG. 8. Ends 20a, 20b are cut on the fibula side after tensioning repair 101 for a final knotless syndesmosis repair. If necessary, the steps can be repeated with a second soft anchor construct (either knotted or knotless anchor) for final fixation. Repair 101 is a knotless repair. Sheath 10 is secured fully within tibia 90 so that the sheath material bunches and deforms within the bone hole, socket, or tunnel, allowing the sheath to anchor in bone 90 and to increase the overall fixation.

Soft anchor construct 100, 200 can be also employed for attachment of soft tissue to bone by passing ends of the flexible coupler through or around soft tissue and forming additional loops around or through soft tissue. One or more flexible couplers 20 and optional shuttling strands may extend through the lumen 12 of sleeve 10 in similar or different directions and/or orientations and/or locations. The flexible tubular sleeve 10 with the flexible couplers and shuttling strands may be secured into or onto bone, and flexible strands can pass over soft tissue (rotator cuff) and are secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

Flexible coupler 20 can be further attached to one or more fixation devices such as any anchors, for example, knotted anchors, knotless anchors, or all-suture anchors, or any devices that confer secure attachment and fixation of soft tissue over bone. The fixation device can be a knotless anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein. The fixation device can also be another all-suture soft anchor like soft anchors 100, 200 detailed above. Flexible coupler 20 can consist essentially of elastic suture.

A soft suture anchor 100, 200 comprises: (i) a cannulated sheath 10 having a length, a first open end 11a, and a second open end 11b; and a flexible coupler 20 attached to the cannulated sheath 10, the flexible coupler 20 having a first free end 20a and a second free end 20b, wherein each of the first free end 20a and the second free end 20b forms a splice 50a, 50b and at least two adjustable, flexible, tensionable loops 50 around and through the cannulated sheath 10. The first free end 20a and the second free end 20b are flexible ends 20a, 20b and are passed through the first open end 11a and the second open end 11b, respectively, and in different directions. The first free end 20a can be passed in a direction of arrow A; the second free end 20b can be passed in a direction of arrow B. The direction of arrow A can be opposite the direction of arrow B. The direction of arrow A can be different from the direction of arrow B. The splice 50a, 50b can be formed outside the sheath 10. Each of the first free end 20a and the second free end 20b can be passed through the first open end 11a and the second open end 11b and through the body of the sheath 10 and entering/exiting the sheath 10 at different locations and with a shuttling device, such as a suture passer.

A method of tissue reconstruction 101, 201 comprises: attaching a flexible coupler 20 to a soft anchor sheath 10 by passing each end 20a, 20b of the flexible coupler 20 through a lumen 12 of the soft anchor sheath 10 and through a body of the soft anchor sheath multiple times and in different directions, to form two splices 50a, 50b and a plurality of adjustable, flexible, tensionable loops 50 around and through the anchor sheath 10, and as part of a soft suture anchor 100; and knotlessly securing the soft suture anchor 100 into bone. The method can further include securing ends 20a, 20b of the flexible coupler 20 to another tissue. The step of securing ends 20a, 20b can be conducted knotlessly. Tissue reconstruction 101 can be a syndesmosis repair. Tissue reconstruction can be an ankle syndesmosis repair. Tissue reconstruction 201 can be a knotless AC repair.

The method can further include securing the flexible coupler 20 to a fixation device. The fixation device can be a knotless anchor. The fixation device can be a hard body anchor. The fixation device can be a soft anchor. The fixation device can be an implantable button. The flexible coupler 20 can consist essentially of elastic suture. A plurality of soft anchors 100, 200 can be used for a soft tissue repair, such as a small joint repair or AC repair.

The constructs and methods of the present disclosure provide increased fixation and consistent longer lasting longer pressure on the tissue.

The constructs of the present disclosure have applicability to any tissue repair and surgical procedure such as, for example, small joint repairs, AC repairs, rotator cuff repairs, Achilles tendon repair, patellar tendon repair, ACL/PCL reconstruction, hip and shoulder reconstruction procedures, and applications for elastic suture used in or with suture anchors. The surgical constructs and repair methods of the present disclosure can be employed in tissue repairs that are knotless but that could also be knotted.

The surgical constructs and repair methods of the present disclosure can be employed in tissue repairs that do not involve knot tying, for example, for use with suture anchors (such as PushLock® and/or SwiveLock® suture anchors) or for knotless arthroscopic suture repairs (such as knotless single row rotator cuff repair, or SpeedBridge™ repairs using no knots and only suture passing steps), among many others.

Flexible coupler 20 can be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided optionally with at least one other elastic fiber, natural or synthetic, to form lengths of suture material. Flexible coupler 20 can also include suture tape such as FiberTape® suture tape (as disclosed in U.S. Pat. No. 7,892,256, the disclosure of which is incorporated in its entirety herewith) or collagen tape, or wide “tape like” material, or combinations thereof.

Flexible coupler 20 can consist essentially of suture material and elastane, or combination of suture material and elastane and other materials such as long chain synthetic polymers like polyester and nylon, or materials such as PET, silk nylon or absorbable polymers, or coating materials (such as wax, silk, or silicone products), among many others. Flexible coupler 20 can consist of strands with cross-sections of various forms and geometries, including round, oval, rectangular, or flat, among others, or combinations of such forms and geometries. In an embodiment, at least one flexible coupler 20 can be provided as a suture which is braided, knitted or woven.

Parts or all of soft anchor 100, 200 including sheath 10 and flexible coupler 20 can be also coated and/or provided in different colors. In an embodiment, parts (or all) of sheath 10 and flexible coupler 20 can be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the construct, pliability, handleability or abrasion resistance, for example.

Parts or all of sheath 10 and/or flexible coupler 20 can be also provided with tinted tracing strands, or otherwise contrast visually with other parts of the construct, which remain a plain, solid color, or displays a different tracing pattern, for example. Various structural elements of the surgical constructs may be visually coded, making identification and handling of the suture legs simpler. Easy identification of suture in situ is advantageous in surgical procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A soft anchor comprising:
a cannulated sheath having a length, a first open end, and a second open end; and
a flexible coupler attached to the cannulated sheath, the flexible coupler having a first free end and a second free end, wherein each of the first free end and the second free end passes through and around the cannulated sheath multiple times to each form a splice and at least two adjustable, flexible, tensionable loops around the cannulated sheath.

2. The soft anchor of claim 1, wherein the first free end enters a first open end of the cannulated sheath at a first location, exits the cannulated sheath at a second location, reenters the cannulated sheath for a second time at a third location, extends within the cannulated sheath, exits the cannulated sheath at the second location, and forms a splice by passing the first free end through itself, wherein the second location is different from the third location.

3. The soft anchor of claim 2, wherein the second free end enters a second open end of the cannulated sheath at a fourth location, exits the cannulated sheath at the third location, reenters the cannulated sheath for a second time at the second location, extends within the cannulated sheath, exits the cannulated sheath at the third location, and forms a splice by passing the second free end through itself, wherein the second location is different from the third location.

4. The soft anchor of claim 3, wherein the second location and the third location are about symmetrically oriented relative to a transversal axis of the cannulated sheath.

5. The soft anchor of claim 3, wherein the second location and the third location are each spaced apart from the first open end and the second open end of the cannulated sheath.

6. The soft anchor of claim 1, wherein the first free end enters a first open end of the cannulated sheath at a first location, exits the cannulated sheath at a second location, reenters the cannulated sheath for a second time at the first open end and at a third location, extends within the cannulated sheath, exits the cannulated sheath at the second location, and forms a splice by passing the first free end through itself, wherein the second location is located at about a middle of the length of the cannulated sheath.

7. The soft anchor of claim 6, wherein the second free end enters a second open end of the cannulated sheath at a fourth location, exits the cannulated sheath at a fifth location, reenters the cannulated sheath for a second time at a sixth location, extends within the cannulated sheath, exits the cannulated sheath at the fifth location, and forms a splice by passing the second free end through itself, wherein the fifth location is located at about a middle of the length of cannulated the sheath.

8. The soft anchor of claim 7, wherein the second location and the fifth location are about symmetrically oriented relative to a transversal axis of the cannulated sheath.

9. The soft anchor of claim 7, wherein the second location and the fifth location are each spaced apart from the first open end and the second open end of the cannulated sheath.

10. The soft anchor of claim 1, wherein at least one region of the flexible coupler is attached to a fixation device.

11. The soft anchor of claim 10, wherein the fixation device is a knotless suture anchor.

12. The soft anchor of claim 1, wherein the soft anchor is an all-suture anchor, and the flexible coupler is round suture.

13. The soft anchor of claim 1, wherein the cannulated sheath is elastic.

14. The soft anchor of claim 1, wherein the soft anchor is manufactured as a one-piece construct.

15. A method of tissue repair comprising:
attaching a flexible coupler to a soft anchor sheath by passing the flexible coupler multiple times through a lumen and a body of the soft anchor sheath to form at least two adjustable, flexible, tensionable loops around the anchor sheath,
wherein the at least two adjustable, flexible, tensionable loops are formed by:
passing a free end of the flexible coupler through an open end of the soft anchor sheath, extending the free end through the lumen of the soft anchor sheath, exiting the free end through the body of the soft anchor sheath, reentering the free end for a second time through the body of the soft anchor sheath, and exiting the soft anchor sheath and forming a splice through itself; and
passing another free end of the flexible coupler through another open end of the soft anchor sheath, extending the another free end through the lumen of the soft anchor sheath, exiting the another free end through the body of the soft anchor sheath, reentering the another free end for a second time through the body of the soft anchor sheath, and exiting the soft anchor sheath and forming a splice through itself.

16. The method of claim 15, further comprising securing the soft anchor sheath into bone.

17. The method of claim 15, further comprising securing the soft anchor sheath at about a mid-length of a bone tunnel.

18. The method of claim 15, wherein the tissue repair is acromioclavicular repair.

19. The method of claim 15, wherein the tissue repair is syndesmosis repair.

20. The method of claim 19, wherein the syndesmosis repair is ankle syndesmosis.

21. The method of claim 15, wherein the tissue repair is a foot, ankle, hand or wrist repair.

22. The method of claim 15, wherein the tissue repair is a knotless repair.
